(19) United States
(12) Patent Application Publication
(10) Pub. No.: US 2002/0072509 A1
(43) Pub. Date: Jun. 13, 2002

(54) Methods for the treatment of a traumatic central nervous system injury

(76) Inventors: Donald Gerald Stein, Atlanta, GA (US); Stuart Wayne Hoffman, Atlanta, GA (US)

Correspondence Address: ALSTON & BIRD LLP, BANK OF AMERICA PLAZA, 101 SOUTH TRYON STREET, SUITE 4000, CHARLOTTE, NC, 28280-4000, US

(21) Appl. No.: 09/973,375

(22) Filed: Oct. 9, 2001

Related U.S. Application Data
(60) Provisional application No. 60/245,798, filed on Nov. 3, 2000.
(60) Provisional application No. 60/239,505, filed on Oct. 11, 2000.

(51) Int. Cl. A61K 31/56

(52) U.S. Cl. 514/169

(57) ABSTRACT

The present invention provides methods for conferring a neuroprotective effect on a population of cells in a subject following a traumatic injury to the central nervous system. Specifically, the methods of the invention provide for the administration of a progestin or progestin metabolite following a traumatic brain injury. The progestin or progestin metabolite is administered at therapeutically effective concentrations that produce a neuroprotective effect (i.e., a decrease in the loss of neuronal activity) and reduces and/or prevents the various physiological events leading to neurodegeneration, such as, cerebral edema and the immune/inflammatory response.

### CROSS-REFERENCE TO RELATED APPLICATIONS

This application claims the benefit of U.S. Provisional Application Nos. 60/245,798, filed Nov. 3, 2000, and 60/239,505, filed Oct. 11, 2000, both of which are herein incorporated by reference in their entirety.

### FEDERALLY SPONSORED RESEARCH OR DEVELOPMENT

This research was funded by the Government Agency Grant R49/CCR412307 and ROI NS38664.

### FIELD OF THE INVENTION

The invention relates to methods for treating a traumatic injury to the central nervous system.

### BACKGROUND OF THE INVENTION

There is growing experimental evidence that progesterone, its metabolites and other gonadal steroids such as estrogen and possibly testosterone, are effective neuroprotective agents; although the specific, physiological mechanisms by which these hormones act in the central nervous system to enhance repair are not completely understood. In addition to being a gonadal steroid, progesterone also belongs to a family of autocrine/paracrine hormones called neurosteroids. Neurosteroids are steroids that accumulate in the brain independently of endocrine sources and which can be synthesized from sterol precursors in nervous cells. These neurosteroids can potentiate GABA transmission, modulate the effects of glutamate, enhance the production of myclin, and prevent release of free radicals from activated microglia.

In vivo data has demonstrated progesterone's neuroprotective effects in injured nervous systems. For example, following a contusion injury, progesterone reduces the severity of post injury cerebral edema. The attenuation of edema by progesterone is accompanied by the sparing of neurons from secondary neuronal death and improvements in cognitive outcome (Roof et al. (1994) Experimental Neurology 129:64-69). Furthermore, following ischemic injury in rats, progesterone has been shown to reduce cell damage and neurological deficit (Jiang et al. (1996) Brain Research 735:101-107). Progesterone's protective effects may be mediated thorough its interaction with GABA and/or glutamate receptors.

Various metabolites of progesterone have also been suggested to have neuroprotective properties. For instance, the progesterone metabolites allopregnanolone or epipregnanolone are positive modulators of the GABA receptor, increasing the effects of GABA in a manner that is independent of the benzodiazepines (Baulieu, E. E. (1992) Adv. Biochem. Psychopharmacol. 47:1-16; Robel et al. (1995) Crit. Rev. Neurobiol. 9:383-94; Lambert et al. (1995) Trends Pharmacol. Sci. 16:295-303; Baulieu, E. E. (1997) Recent Prog. Horm. Res. 52:1-32; Reddy et al. (1996) Psychopharmacology 128:280-92). In addition, these neurosteroids act as antagonists at the sigma receptor: a receptor that can activate the NMDA channel complex (Maurice et al. (1998) Neuroscience 83:413-28; Maurice et al. (1996) J. Neurosci. Res. 46:734-43; Reddy et al. (1998) Neuroreport 9:3069-73). These neurosteroids have also been shown to reduce the stimulation of cholinergic neurons and the subsequent release of acetylcholine by excitability Numerous studies have shown that the cholinergic neurons of the basal forebrain are sensitive to traumatic brain injury and that excessive release of acetylcholine can be more excitotoxic than glutamate (Lyeth et al. (1992) J. Neurotrauma 9(2):S463-74; Hayes et al. (1992) J. Neurotrauma 9(1):S173-87).

Following a traumatic injury to the central nervous system, a cascade of physiological events leads to neuronal loss including, for example, an inflammatory immune response and excitotoxicity resulting from the initial impact disrupting the glutamate, acetylcholine, cholinergic, GABA_{A}, and NMDA receptor systems. In addition, the traumatic CNS injury is frequently followed by brain and/or spinal cord edema that enhances the cascade of injury and leads to further secondary cell death and increased patient mortality. Methods are needed for the in vivo treatment of traumatic CNS injuries that are successful at providing subsequent trophic support to remaining central nervous system tissue, and thus enhancing functional repair and recovery, under the complex physiological cascade of events which follow the initial insult.

### SUMMARY OF THE INVENTION

Methods for the treatment or the prevention of neuronal damage in the CNS are provided. In particular, the present invention provides a method for treating or preventing neuronal damage caused by a traumatic injury to the CNS through the administration of a therapeutically effective concentration of progesterone or a progestin metabolite. In one embodiment, the present invention provides a method of treating a traumatic brain injury resulting from a blunt force contusion. In other embodiments, the present invention provides a method of reducing cerebral edema and/or the inflammatory response in a patient following a traumatic brain injury. The methods of the invention further encompass the reduction of neuronal cell death in a patient following a traumatic brain injury by the administration of the progestin metabolite.

### BRIEF DESCRIPTION OF THE DRAWINGS

FIG. 1 shows the level of reduction of cerebral edema by progesterone (p), epipregnanolone (EP), allopregnanolone (AP), vehicle (Veh) and sham operates at 2, 24, and 72 hours post injury.

FIG. 2 shows that administration of allopregnanolone following a cortical contusion injury results in an improved performance on an acquisition task in the Morris Water Maize. A single asterisk (*) indicates a difference between injured rat given vehicles and those treated with allopregnanolone (p<0.05).

FIG. 3 shows the results from a histological analysis of the number of CHAT positive cells in the nucleus basalis magnocellularis following a bilateral frontal cortical contusion and subsequent treatment with progesterone (LP), allopregnanolone (LAP), epipregnanolone (LEP), sham-vehicle (Sham), and injury vehicle (Lesion). A single asterisk (*) indicates a significant difference from sham, a double asterisk (**) indicates a significant difference from both sham and lesion controls.

FIG. 4 shows the level of TNF mRNA expression in brain tissue following a bilateral frontal cortical contusion. Neurosteroid injections were given at 1 hr and 6 hrs after the contusions, and continued once a day for up to 5 consecutive days post-injury. At 3 hrs, 8 hrs, 12 hrs, and 6 days post-injury, the level of TNF mRNA expression was determined. sham-vehicle (SV); sham-progesterone (SP); sham-allopregnanolone (SA); lesion-vehicle (LV); Lesion-progesterone (LP); lesion-allopregnanolone. *=P<0.05

FIG. 5 shows the level of IL-1 mRNA expression in brain tissue following a bilateral frontal cortical contusion. Neurosteroid injections were given at 1 hr and 6 hrs after the contusions, and continued once a day for up to 5 consecutive days post-injury. At 3 hrs, 8 hrs, 12 hrs and 6 days post-injury, the level of IL-1 mRNA expression was determined. sham-vehicle (SV); sham-progesterone(SP); sham-allopregnanolone (SA); lesion-vehicle (LV); Lesion-progesterone (LP); lesion-allopregnanolone. *=P<0.05

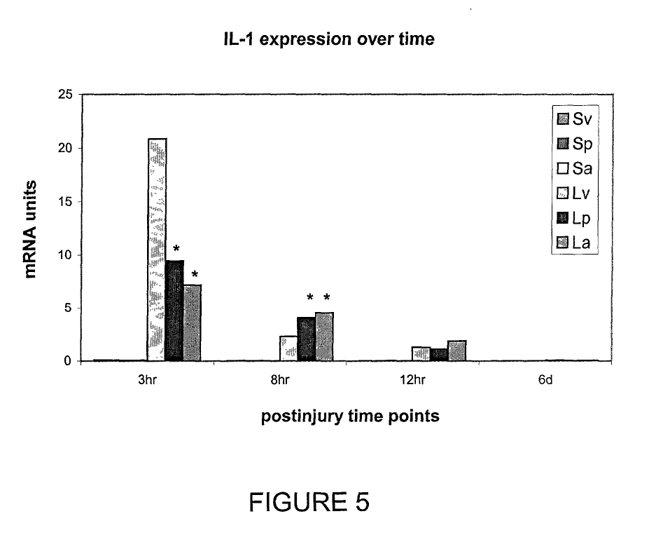

FIG. 6 shows a dosage response curve for behavioral recovery following a traumatic brain injury. FIGS. 6A and 6B demonstrate that following treatment with low (8 mg/kg), moderate (16 mg/kg), and high (32 mg/kg) doses of progesterone in a cyclodextrin-containing carrier, both low and moderate doses of progesterone produced consistent improvement in Morris water maze performance.

FIG. 7 shows the results from the “sticker removal task” following treatment with low (8 mg/kg), moderate (16 mg/kg), and high (32 mg/kg) dosages of progesterone in a cyclodextrin-containing carrier.

### DETAILED DESCRIPTION OF THE INVENTION

The present invention provides methods and compositions for the treatment or prevention of neurodegeneration following a traumatic injury to the central nervous system. By “treatment or prevention” is intended any enhanced survival, proliferation, and/or neurite outgrowth of the neurons that either prevents or retards neurodegeneration. Neurodegeneration is the progressive loss of neurons in the central nervous system. As used herein, “neuroprotection” is the arrest and/or reverse progression of neurodegeneration following a traumatic central nervous system injury. The neuroprotective effect includes both improved morphological (i.e., enhanced tissue viability) and/or behavioral recovery. The improvement can be characterized as an increase in either the rate and/or the extent of behavioral and anatomical recovery following the traumatic CNS injury. In the methods of the present invention, neuroprotection following a traumatic CNS injury is achieved by the administration of a therapeutically effective composition comprising a progesterone or a progestin metabolite to a patient (i.e., a mammal, preferably a human).

Multiple physiological events lead to the neurodegeneration of the CNS tissues following a traumatic CNS injury. These events include, for example, cerebral edema, destruction of vascular integrity, increase in the immune and inflammatory response, demyelinization, and lipid peroxidation. Hence, the methods of the invention also find use in reducing and/or preventing the physiological events leading to neurodegeneration. Specifically, the present invention provides methods for reducing or eliminating neuronal cell death, edema, ischemia, and enhancing tissue viability following a traumatic injury to the central nervous system.

The sex hormones are steroids that may be classified into functional groups according to chemical structure and physiological activity and include estrogenic hormones, progestational hormones, and androgenic hormones. Of particular interest in the methods of the present invention are progestational hormones, referred to herein as “progestins” or “progestogens”, and their derivatives and bioactive metabolites. Members of this broad family include steroid hormones disclosed in Remingtons' Pharmacueutical Sciences, Gennaro et al., Mack Publishing Co. (18^{th} ed. 1990), 990-993. As with all other classes of steroids, sterioisomerism is of fundamental importance with the sex hormones. Hence, a variety of progestins (i.e., progesterone) and their derivatives are encompassed by the present invention, including both synthetic and natural products. As used herein, by “bioactive metabolite” or “derivative” of progestin is intended any naturally or synthetically produced progestin that prevents or retards neurodegeneration. Such progestin derivatives include, for example, derivatives of progesterone, such as 5-dehydroprogesterone, 6-dehydro-retroprogesterone (dydrogesterone), allopregnanolone (allopregnan-3α, or 3β-ol-20-one), ethynodiol diacetate, hydroxyprogesterone caproate (pregn-4-ene-3,20-dione, 17-(1-oxohexy)oxy); levonorgestrel, norethindrone, norethindrone acetate (19-norpregn-4-en-20-yn-3-one, 17-(acetyloxy)-,(17α)-); norethynodrel, norgestrel, pregnenolone, and megestrol acetate. Useful progestins also can include allopregnone-3α or 3β, 20α or 20β-diol (see Merck Index 258-261); allopregnane-3β,21-diol-11,20-dione; allopregnane-3β,17α-diol-20-one; 3,20-allopregnanedione, allopregnane,3β,11β,17α,20β,21-pentol; allopregnane-3β,17α,20β,21-tetrol; allopregnane-3α or 3β,11β,17α,21-tetrol-20-one, allopregnane-3β,17α or 20β-triol; allopregnane-3β,17α,21-triol-11,20-dione; allopregnane-3β,11β,21-triol-20-one; allopregnane-3β,17α,21-triol-20-one; allopregnane-3α or 3β-ol-20-one; pregnanediol; 3,20-pregnanedione; pregnan-3α-ol-20-one; 4-pregnene-20,21-diol-3,11-dione; 4-pregnene-11β,17α,20β,21-tetrol-3-one; 4-pregnene-17α,20β,21-triol-3,11-dione; 4-pregnene-17α,20β,21-triol-3-one, and pregnenolone methyl ether. Further progestin derivatives include esters with non-toxic organic acids such as acetic acid, benzoic acid, maleic acid, malic acid, caproic acid, citric acid and the like. Inorganic salts include, for example, hydrochloride, sulfate, nitrate, bicarbonate and carbonate salts. Additionally, compounds that may find use in the present invention include the progestin derivatives that are disclosed in U.S. Pat. No. 5,232,917, herein incorporated by reference.

The present invention provides a method to achieve a neuroprotective effect following a traumatic CNS injury in a patient (i.e., a mammal, preferably a human) through the administration of a therapeutically effective composition comprising at least one progestin or a progestin metabolite. A traumatic injury to the CNS is characterized by a physical impact to the central nervous system. For example, a traumatic brain injury results when the brain is subjected to a physical force that results in progressive neuronal cell damage and/or cell death. A traumatic brain injury may result from a blow to the head and manifests as either an open or closed injury. Severe brain damage can occur from lacerations, skull fractures, and conversely, even in the absence of external signs of head injury. The physical forces resulting in a traumatic brain injury cause their effects by inducing three types of injury: skull fracture, parenchymal injury, and vascular injury.

Parenchymal injuries include concussion, direct parenchymal injury and diffuse axonal injury. Concussions are characterized as a clinical syndrome of alteration of consciousness secondary to head injury typically resulting from a change in the momentum of the head (movement of the head arrested against a ridged surface). The pathogenesis of sudden disruption of nervous activity is unknown, but the biochemical and physiological abnormalities that occur include, for example, depolarization due to excitatory amino acid-mediated ionic fluxes across cell membranes, depletion of mitochondrial adenosine triphosphate, and alteration in vascular permeability. Postconcussive syndrome may show evidence of direct parenchymal injury, but in some cases there is no evidence of damage.

Contusion and lacerations are conditions in which direct parenchymal injury of the brain has occurred, either through transmission of kinetic energy to the brain and bruising analogous to what is seen in soft tissue (contusion) or by penetration of an object and tearing of tissue (laceration). A blow to the surface of the brain leads to rapid tissue displacement, disruption of vascular channels, and subsequent hemorrhage, tissue injury and edema. Morphological evidence of injury in the neuronal cell body includes pyknosis of nucleus, eosinophilia of the cytoplasm, and disintegration of the cell. Furthermore, axonal swelling can develop in the vicinity of damage neurons and also at great distances away from the site of impact. The inflammatory response to the injured tissue follows its usual course with neutrophiles preceding the appearance of macrophages.

The methods of the present invention find use in producing a neuroprotective effect following a traumatic injury to the central nervous system. Methods to quantify the extent of central nervous system damage (i.e., neurodegeneration) and to determine if neuronal damage was treated or prevented following the administration of a progesterone or progesterone metabolite are well known in the art. Such neuroprotective effects can be assayed at various levels, including, for example, by promoting behavioral and morphological (i.e., enhancing tissue viability) recovery after traumatic brain injury. A variety of anatomical, immunocytochemical and immunological assays to determine the effect of the progestin metabolite on necrosis, apoptosis, and neuronal glial repair are known in the art. As such, the neuroprotection resulting from the methods of the present invention will result in at least about a 10% to 20%, 20% to 30%, 30% to 40%, 40% to 60%, 60% to 80% or greater increase in neuronal survival and/or behavioral recovery as compared to the control groups.

Histological and molecular marker assays for an increase in neuronal survival are known. For example, Growth Associated Protein 43 (GAP-43) can be used as a marker for new axonal growth following a CNS insult. See, for example, Stroemer et al. (1995) Stroke 26:2135-2144, Vaudano et al. (1995) J. of Neurosci 15:3594-3611. Other histological markers can include a decrease in astrogliosis and microgliosis. Alternatively, a delay in cellular death can be assayed using TUNEL labeling in injured tissue. Further anatomical measures that can be used to determine an increase in neuroprotection include counting specific neuronal cell types to determine if the progestin or the progestin metabolite is preferentially preserving a particular cell type (e.g., cholinergic cells) or neurons in general.

In addition, behavioral assays can be used to determine the rate and extent of behavior recovery in response to the treatment. Improved patient motor skills, spatial learning performance, cognitive function, sensory perception, speech and/or a decrease in the propensity to seizure may also be used to measure the neuroprotective effect. Such functional/behavioral tests used to assess sensorimortor and reflex function are described in, for example, Bederson et al. (1986) Stroke 17:472-476, DeRyck et al. (1992) Brain Res. 573:44-60, Markgraf et al. (1992) Brain Res. 575:238-246, Alexis et al. (1995) Stroke 26:2336-2346; all of which are herein incorporated by reference. Enhancement of neuronal survival may also be measured using the Scandinavian Stroke Scale (SSS) or the Barthl Index. Behavioral recovery can be further assessed using the recommendations of the Subcommittee of the NIH/NINDS Head Injury Centers in Humans (Hannay et al. (1996) J. Head Trauma Rehabil. 11:41-50), herein incorporated by reference. Behavioral recovery can be further assessed using the methods described in, for example, Beaumont et al. (1999) Neurol Res. 21:742-754; Becker et al. (1980) Brain Res. 200:07-320; Buresov et al. (1983) Techniques and Basic Experiments for the Study of Brain and Behavior; Kline et al. (1994) Pharmacol. Biochem. Behav. 48:773-779; Lindner et al. (1998) J. Neurotrauma 15:199-216; Morris (1984) J. Neurosci. Methods 11:47-60; Schallert et al. (1983) Pharmacol. Biochem. Behav. 18:753-759.

It is recognized that a traumatic injury to the CNS results in multiple physiological events that impact the extent and rate of neurodegeneration, and thus the final clinical outcome of the injury. The treatment of a traumatic injury to the CNS, as defined by the present invention, encompasses any reduction and/or prevention in one or more of the various physiological events that follow the initial impact. Hence, the methods of the invention find use in the physiological events leading to neurodegeneration following a traumatic injury to the central nervous system.

For instance, cerebral edema frequently develops following a traumatic injury to the CNS and is a leading cause of death and disability. Cortical contusions, for example, produce massive increases in brain tissue water content which, in turn, can cause increased intracranial pressure leading to reduced cerebral blood flow and additional neuronal loss. Hence, the methods of the invention find use in reducing and/or eliminating cerebral edema and/or reducing the duration of the edemic event following a traumatic injury to the CNS. Assays to determine a reduction in edema are known in the art and include, but are not limited to, a decrease in tissue water content following the administration of the progestin or the progestin metabolite (Betz et al. (1990) Stroke 21:1199-204, which is herein incorporated by reference). Furthermore, an overall improvement in behavioral recovery can also be used as a measure for a decrease in edema. A decrease in edema in the effected tissue by at least about 15% to 30%, about 30% to 45%, about 45% to 60%, about 60% to 80%, or about 80% to 95% or greater will be therapeutically beneficial, as will any reduction in the duration of the edemic event

Vasogenic edema following a traumatic brain injury has been associated with damage to the vasculature and disruption of the blood-brain barrier (BBB) (Duvdevani et al. (1995) J. Neurotrauma 12:65-75, herein incorporated by reference). Progesterone has been shown to reduce the permeability of the BBB to macromolecules, but not ions, such as sodium in vitro (Betz et al. (1990) Stroke 21:1199-204; Beta et al. (1990) Acta. Neurochir. Suppl. 51:256-8; both of which are herein incorporated by reference). Hence, the methods of the invention find use in reducing or eliminating vasogenic edema following a traumatic brain injury. Assays to determine a decrease in vasogenic edema are known in the art and include, for instance, a reduction in Evans' blue extravasation after cortical contusion (Roof et al. (1994) Society for Neuroscience 20:91, herein incorporated by reference).

Further physiological effects of a traumatic brain injury include an immune response. See, for example, Soares et al. (1995) J. Neurosci. 15:8223-33; Holmin et al. (1995) Acta Neurochir. 132:110-9; Arvin et al. (1996) Neurosci. Biobehav. Rev. 20:445-52. Following a cortical impact, severe inflammatory reactions and gliosis at the impact site and at brain areas distal to the primary site of injury occurs. The inflammatory response is characterized by the expression of adhesion molecules on the vascular surfaces, resulting in the adherence of immune cells and subsequent extravasation into the brain parenchyma. By releasing cytokines, the invading macrophages and neutrophils stimulate reactive astrocytosis. Release of different chemokines by other cell types induces these immune cells to become phagocytic, with the simultaneous release of free radicals and pro-inflammatory compounds, e.g., cytokines, prostaglandins, and excitotoxins (Arvin et al. (1996) Neurosci. Biobehav. Ref. 20:445-52; Raivich et al. (1996) Kelo J. Med. 45:239-47; Mattson et al. (1997) Brain Res. Rev. 23:47-61; all of which are herein incorporated by reference).

The methods of the invention provide a means to reduce or eliminate the inflammatory immune reactions that follow a traumatic CNS injury. Furthermore, by reducing the inflammatory response following an injury, the progestin or progestin metabolite of the present invention can substantially reduce brain swelling and intracranial pressure and reduce the amount of neurotoxic substances (e.g., free radicals and excitotoxins) that are released. Therefore, by reducing the immune/inflammatory response following a traumatic injury to the CNS, neuronal survival and/or behavioral recovery will be enhanced.

Assays that can be used to determine if the progestin metabolite of the invention is imparting an anti-inflammatory and a nonspecific suppressive effect on the immune system following a traumatic CNS injury include, for example, a reduction in cytokine induced microglial proliferation in vitro (Hoffman et al. (1994) J. Neurotrauma 11:417-31; Garcia-Estrada et al. (1993) Brain Res. 628:271-8; both of which are herein incorporated by reference); a reduction in the generation of cytotoxic free radicals by activated macrophages (Chao et al. (1994) Am. J. Reprod. Immunol. 32:43-52; Robert et al. (1997) Nitric Oxide 1:453-62; Kelly et al. (1997) Biochem. Biophys. Res. Commun. 239:557-61; Ganter et al. (1992) J. Neurosci. Res. 33:218-30; all of which are herein incorporated by reference); a reduction in the expression of inducible nitric oxide synthetase and the amount of nitric oxide release by macrophages (Robert et al. (1997) Nitric Oxide 1:453-62; Miller et al (1996) J. Leukoc. Biol. 59:442-50; both of which are herein incorporated by reference); the release of a “progesterone-induced blocking factor” that inhibits natural killer cell activity (Cheek et al. (1997) Am. J. Reprod. Immunol. 37:17-20; Szekeres-Bartho et al. (1997) Cell Immunol. 177:194-9; Szekeres-Bartho et al. (1996) Am. J. Reprod. Immunol. 35:348-51; all of which are herein incorporated by reference); a decrease in the number of GFAP-positive astrocytes after brain injury which is suggestive of less secondary damage (Garcia-Estrada et al. (1993) Brain Res. 628:271-8; Garcie-Estrada et al. (1999) Int. J. Dev. Neurosci. 17:145-51; Cheek et al. (1997) Am. J. Reprod. Immunol. 37:17-20; Szekeres-Bartho et al. (1997) Cell Immunol. 177:194-9; Szekeres-Bartho et al. (1996) Am. J. Reprod. Immunol. 35:348-51; all of which are herein incorporated by reference); a reduction in the number of inflammatory immune cells (OX42-positive cells); a reduction in the loss of ChAT-positive and COX-positive neurons; a reduction in the number of TUNEL-positive and MnSOD-positive neurons; and an increase in the intensity of succinate dehydrogenase and cytochrome oxidase activity.

Furthermore, a reduction in the inflammatory immune reactions following a traumatic brain injury can be assayed by measuring the cytokines level following the injury in the sham controls versus the progestin treated subjects. Cytokines are mediators of inflammation and are released in high concentrations after brain injury. The level of pro-inflammatory cytokines (e.g., interleukin 1-beta, tumor necrosis factor, and interleukin 6) and the level of anti-inflammatory cytokines (e.g., interleukin 10 and transforming growth factor-beta) can be measured. For instance, “real-time” polymerase chain reactions (PCR) can be used to measure the strength of the mRNA signal and ELISA can be used to determine protein levels. In addition, histological analysis for different inflammatory cell types (e.g., reactive astrocytes, macrophages and microglia) can be used to measure a reduction in the inflammatory response.

The methods of the invention may also be used to decrease ischemia following a traumatic brain injury. Assays for a decrease in an ischemic event include, for example, a decrease in infarct area, improved body weight, and improved neurological outcome.

Another physiological consequence of a traumatic CNS injury is an increase in lipid peroxidiation. The methods of the invention find use in reducing free radical damage and thus decreasing or eliminating lipid peroxidation. This effect may occur through an enhancement of endogenous free radical scavenging systems. Assays to measure a reduction in lipid peroxidation in both brain homogenate and in mitochondria are known in the art and include, for example, the thiobarbituric acid method (Roof et al. (1997) Mol. Chem. Neuropathol. 31:1-11; Subramanian et al. (1993) Neurosci. Lett. 155:151-4; Goodman et al. (1996) J. Neurochem. 66:1836-44; Vedder et al. (1999) J. Neurochem. 72:2531-8; all of which are herein incorporated by reference) and various in vitro free radical generating systems Furthermore, alterations in the levels of critical free radical scavenger enzymes, such as mitochondrial glutathione can be assayed. See, for example, Subramanian et al. (1993) Neurosci. Lett. 155:151-4; and Vedder et al. (1999) J. Neurochem. 72:2531-8; both of which are herein incorporated by reference.

Furthermore, cultured, cytokine-stimulated macrophages generate nitrite, superoxide, and hydrogen peroxide. Since macrophages are known to be very active between 48 hours and seven days after a traumatic brain injury, a reduction in these reactive cells would reduce secondary damage to neurons. See, for example, Fulop et al. (1992) 22^{nd} Annual Meeting of the Society for Neuroscience 18:178; Soares et al. (1995) J. Neurosci. 15:8223-33; Holmin et al. (1995) Acta Neurochir. 132:110-9; all of which are herein incorporated by reference.

The present invention provides for a method of treating a traumatic brain injury by administering to a subject a progestin or derivative thereof in a therapeutically effective amount. By “therapeutically effective amount” is meant the concentration of a progestin or progestin metabolite that is sufficient to elicit a therapeutic effect. Thus, the concentration of a progestin or progestin metabolite in an administered dose unit in accordance with the present invention is effective in the treatment or prevention of neuronal damage that follows a traumatic injury to the CNS and hence, elicits a neuroprotective effect. The therapeutically effective amount will depend on many factors including, for example, the specific activity of the progestin or progestin metabolite, the severity and pattern of the traumatic injury, the resulting neuronal damage, the responsiveness of the patient, the weight of the patient along with other intraperson variability, the method of administration, and the progestin or progestin formulation used. Methods to determine efficacy, dosage, and route of administration are known to those skilled in the art.

The progestin or progestin metabolite employed in the methods of the invention may further comprise an inorganic or organic, solid or liquid, pharmaceutically acceptable carrier. The carrier may also contain preservatives, wetting agents, emulsifiers, solubilizing agents, stabilizing agents, buffers, solvents and salts. Compositions may be sterilized and exist as solids, particulants or powders, solutions, suspensions or emulsions.

The progestin or progestin metabolites can be formulated according to known methods to prepare pharmaceutically useful compositions, such as by admixture with a pharmaceutically acceptable carrier vehicle. Suitable vehicles and their formulation are described, for example, in Remington's Pharmaceutical Sciences (16th ed., Osol, A. (ed.), Mack, Easton, Pa. (1980)). In order to form a pharmaceutically acceptable composition suitable for effective administration, such compositions will contain an effective amount of the progestin or progestin metabolite, either alone, or with a suitable amount of carrier vehicle.

The pharmaceutically acceptable carrier of the present invention will vary depending on the method of drug administration. The pharmaceutical carrier employed may be, for example, either a solid, liquid, or time release. Representative solid carriers are lactose, terra alba, sucorse, talc, geletin, agar, pectin, acacia, magnesium stearate, stearic acid, microcrystalin cellulose, polymer hydrogels, and the like. Typical liquid carriers include syrup, peanut oil, olive oil, cyclodextrin, and the like emulsions. Those skilled in the art are familiar with appropriate carriers for each of the commonly utilized methods of administration. Furthermore, it is recognized that the total amount of progestin or progestin administered as a therapeutic effective dose will depend on both the pharmaceutical composition being administered (i.e., the carrier being used) and the mode of administration.

An embodiment of the present invention provides for the administration of a progestin metabolite or analogue thereof via parenteral administration in a dose of about 0.1 ng to about 100 g per kg of body weight, about 10 ng to about 50 g per kg of body weight, from about 100 ng to about 1 g per kg of body weight, from about 1 μg to about 100 mg per kg of body weight, from about 1 μg to about 50 mg per kg of body weight, from about 1 mg to about 500 mg per kg of body weight; and from about 1 mg to about 50 mg per kg of body weight. Alternatively, the amount of progestin metabolite administered to achieve a therapeutic effective dose is about 0.1 ng, 1 ng, 10 ng, 100 ng, 1 μg, 10 μg, 100 μg, 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 30 mg, 40 mg, 50 mg, 60 mg, 70 mg, 80 mg, 90 mg, 100 mg, 500 mg per kg of body weight or greater.

Administration of the progestin or progestin metabolite of the invention may be performed by many methods known in the art. The present invention comprises all forms of dose administration including, but not limited to, systemic injection, parenteral administration, intravenous, intraperitoneal, intramuscular, transdermal, buccal, subcutaneous and intracerebroventricular administration. Alternatively, the progestin or progestin metabolite may be administered directly into the brain or cerebrospinal fluid by any intracerebroventricular technique including, for example, lateral cerebro ventricular injection, lumbar puncture or a surgically inserted shunt into the cerebro ventricle of a patient. Methods of administering may be by dose or by control release vehicles.

While the methods of the invention are not bound by any theory, it is believed that a traumatic CNS injury, may make the blood/brain barrier more permeable allowing entry of large molecules that would not normally cross the blood/brain barrier to enter the cerebral spinal fluid. For examples of intravenous, intraperitoneal, intramuscular, and subcutaneous administration of neurotrophic agents to treat CNS injuries see, for example, U.S. Pat. No. 5,733,871 and WO 97/21449 both of which are herein incorporated by reference.

Additional pharmaceutical methods may be employed to control the duration of action. Controlled release preparations may be achieved by the use of polymers to complex or absorb the progestin metabolite. The controlled delivery may be exercised by selecting appropriate macromolecules (for example, polyesters, polyamino acids, polyvinyl pyrrolidone, ethylene-vinylacetate, methylcellulose, carboxymethylcellulose, or protamine sulfate). The rate of drug release may also be controlled by altering the concentration of such macromolecules.

Another possible method for controlling the duration of action comprises incorporating the therapeutic agents into particles of a polymeric substance such as polyesters, polyamino acids, hydrogels, poly(lactic acid) or ethylene vinylacetate copolymers. Alternatively, it is possible to entrap the therapeutic agents in microcapsules prepared, for example, by coacervation techniques or by interfacial polymerization, for example, by the use of hydroxymethyl cellulose or gelatin-microcapsules or poly(methylmethacrylate) microcapsules, respectively, or in a colloid drug delivery system, for example, liposomes, albumin, microspheres, microemulsions, nanoparticles, nanocapsules, or in macroemulsions. Such teachings are disclosed in Remington's Pharmaceutical Sciences (1980).

In further embodiments of the present invention, at least one additional neuroprotective agent can be combined with the progestin metabolite to enhance neuroprotection following a traumatic CNS injury. Such agents include, any combination of a progestin derivative thereof Other neuroprotective agents of interest include, for example, compounds that reduce glutamate excitotoxicity and enhance neuronal regeneration. Such agents may be selected from, but not limited to, the group comprising growth factors. By “growth factor” is meant an extracellular polypeptide-signaling molecule that stimulates a cell to grow or proliferate. Preferred growth factors are those to which a broad range of cell types respond. Examples of neurotrophic growth factors include, but are no limited to, fibroblast growth factor family members such as basic fibroblast growth factor (bFGF) (Abraham et al. (1986) Science 233:545-48), acidic fibroblast growth factor (aFGF) (Jaye et al. (1986) Science 233:541-45), the hst/Kfgf gene product, FGF-3 (Dickson et al. (1987) Nature 326-833), FGF-4 (Zhan et al. (1988) Mol. Cell. Biol. 8:3487-3495), FGF-6 (deLapeyriere et al. (1990) Oncogene 5:823-831), keratinocyte growth factor (KGF) (Finch et al. (1989) Science 245:752-755), and androgen-induced growth factor (AIGF) (Tanaka et al. (1992) Proc. Natl. Acad. Sci. USA 89:8928-8923).

Additional neuroprotective agents include, ciliary neurotrophic factor (CNTF), nerve growth factor (NGF) (Seiler, M. (1984) Brain Research 300:33-39; Hagg T. et al. (1988) Exp Neurol 101:303-312; Kromer L. F. (1987) Science 235:214-216; and Hagg T. et al. (1990) J. Neurosci 10(9):3087-3092), brain derived neurotrophic factor (BDNF) (Kiprianova, I. et al. (1999) J. Neurosci. Res. 56:21-27), Neurotrophin 3 (NT3), Neurotrophin 4 (NT4), transforming growth factor-β1 (TGF-β1) (Henrick-Noack, P. et al. (1996) Stroke 27:1609-14), bone morphogenic protein (BMP-2) (Hattori, A. et al. (1999) J. Neurochem. 72:2264-71), glial-cell line derived neurotrophic factor (GDNF) (Miyazaki, H. et al. (1999) Neuroscience 89:643-7), activity-dependant neurotrophic factor (ADNF) (Zamostiano, R. et al. (1999) Neurosci Letter 264:9-12), cytokine leukemia inhibiting factor (LIF) (Blesch, A. et al. (1999) J. Neurosci. 19:3356-66), oncostatin M, interleukin, and the insulin-like growth factors 1 and 2.

Other forms of neuroprotective therapeutic agents include, for example, Clomethiazole (Zendra) (Marshal, J. W. et al. (1999) Exp. Neurol. 156:121-9); kynurenic acid (KYNA) (Salvati, P. et al. (1999) Prog Neruopsychopharmacol Biol Psychiatry 23:741-52), Semax (Miasoedova, N. F. et al. (1999) Zh Nevrol Psikhiatr Imss Korsakova 99:15-19), FK506 (tacrolimus) (Gold, B. G. et al. (1999) J. Pharmacol. Exp. Ther. 289:1202-10), L-threo-1-phenyl-2-decanoylamino-3-morpholino-1-propanol (Inokuchi, J. et al. (1998) Act Biochim Pol 45:479-92), andrenocorticotropin-(4-9) analoge (ORG 2766) and dizolcipine (MK-801) (Herz, R. C. et al. (1998) Eur J. Pharmacol 346:159-65), cerebral interleukin-6) (Loddick, S. A. et al. (1998) J. Cereb Blood Flow Metab 18:176-9), selegiline (Semkova, I. et al. (1996) Eur J. Pharmacol 315:19-30), MK-801 (Barth, A. et al. (1996) Neuro Report 7:1461-4; glutamate antagonist such as, NPS1506, GV1505260, MK801 (Baumgartner, W. A. et al.(1999) Ann Thorac Surg 67:1871-3), GV150526 (Dyker, A. G. et al. (1999) Stroke 30:986-92); AMPA antagonist such as NBQX (Baumgartner, W. A. (1999) et al. Ann Thorac Surg 67:1871-3, PD152247 (PNQX) (Schielke, G. P. et al. (1999) Stroke 30:1472-7), SPD 502 (Nielsen, E. O. et al. (1999) J. Pharmacol Exp Ther 289:1492-501), LY303070 and LY300164 (May, P. C. et al. (1999) Neuroscience Lett 262:219-221).

When the progestin or progestin metabolite of the present invention is administered conjointly with other pharmaceutically active agents, (i.e., other neuroprotective agents) even less of the progestin metabolite may be therapeutically effective. The progestin metabolite may be administered once or several times a day. The duration of the treatment may be once per day for a period of from two to three weeks and may continue for a period of months or even years. The daily dose can be administered either by a single dose in the form of an individual dosage unit or several smaller dosage units or by multiple administration of subdivided dosages at certain intervals.

For instance, a dosage unit can be administered from 0 hours to 1 hr, 1 hr to 24 hr or 24 hours to at least 100 hours post injury. Alternatively, the dosage unit can be administered from about 0.5, 1, 2, 3, 4, 5, 6, 7, 8, 9, 10, 11, 12, 13, 14, 15, 16, 17, 18, 19, 20, 21, 22, 23, 24, 30, 40, 48, 72, 96, 120 hours or longer post injury. Subsequent dosage units can be administered any time following the initial administration such that a therapeutic effect is achieved. For instance, additional dosage units can be administered to protect the subject from the secondary wave of edema that may occur over the first several days post-injury.

The progestin or progestin metabolite may be administered per se or in the form of a pharmaceutically acceptable salt. When used in medicine, the salts of the progestin metabolite should be both pharmacologically and pharmaceutically acceptable, but non-pharmaceutically acceptable salts may conveniently be used to prepare the free active compound or pharmaceutically acceptable salts thereof and are not excluded from the scope of this invention. Such pharmacologically and pharmaceutically acceptable salts can be prepared by reaction of a progestin metabolite with an organic or inorganic acid, using standard methods detailed in the literature. Examples of pharmaceutically acceptable salts are organic acids salts formed from a physiologically acceptable anion, such as, tosglate, methenesulfurate, acetate, citrate, malonate, tartarate, succinate, benzoate, etc. Inorganic acid salts can be formed from, for example, hydrochloride, sulfate, nitrate, bicarbonate and carbonate salts. Also, pharmaceutically acceptable salts can be prepared as alkaline metal or alkaline earth salts, such as sodium, potassium, or calcium salts of the carboxylic acid group.

Thus the present invention also provides pharmaceutical formulations or compositions, both for veterinary and for human medical use, which comprise the a progestin metabolite or a pharmaceutically acceptable salt thereof with one or more pharmaceutically acceptable carriers thereof and optionally any other therapeutic ingredients, such as other neurotrophic agents. The carrier(s) must be pharmaceutically acceptable in the sense of being compatible with the other ingredients of the formulation and not unduly deleterious to the recipient thereof.

The compositions includes those suitable for oral, rectal, topical, nasal, ophthalmic, or parenteral (including intraperitoneal, intravenous, subcutaneous, or intramuscular injection) administration. The compositions may conveniently be presented in unit dosage form and may be prepared by any of the methods well known in the art of pharmacy. All methods include the step of bringing the active agent into association with a carrier that constitutes one or more accessory ingredients. In general, the compositions are prepared by uniformly and intimately bringing the active compound into association with a liquid carrier, a finely divided solid carrier or both, and then, if necessary, shaping the product into desired formulations.

Compositions of the present invention suitable for oral administration may be presented as discrete units such as capsules, cachets, tablets, lozenges, and the like, each containing a predetermined amount of the active agent as a powder or granules; or a suspension in an aqueous liquor or non-aqueous liquid such as a syrup, an elixir, an emulsion, a draught, and the like.

A tablet may be made by compression or molding, optionally with one or more accessory ingredients. Compressed tablets may be prepared by compressing in a suitable machine, with the active compound being in a free-flowing form such as a powder or granules which is optionally mixed with a binder, disintegrant, lubricant, inert diluent, surface active agent or dispersing agent. Molded tablets comprised with a suitable carrier may be made by molding in a suitable machine.

A syrup may be made by adding the active compound to a concentrated aqueous solution of a sugar, for example sucrose, to which may also be added any accessory ingredient(s). Such accessory ingredients may include flavorings, suitable preservatives, an agent to retard crystallization of the sugar, and an agent to increase the solubility of any other ingredient, such as polyhydric alcohol, for example, glycerol or sorbitol.

Formulations suitable for parental administration conveniently comprise a sterile aqueous preparation of the active compound, which can be isotonic with the blood of the recipient.

Nasal spray formulations comprise purified aqueous solutions of the active agent with preservative agents and isotonic agents. Such formulations are preferably adjusted to a pH and isotonic state compatible with the nasal mucous membranes.

Formulations for rectal administration may be presented as a suppository with a suitable carrier such as cocoa butter, or hydrogenated fats or hydrogenated fatty carboxylic acids.

Ophthalmic formulations are prepared by a similar method to the nasal spray, except that the pH and isotonic factors are preferably adjusted to match that of the eye.

Topical formulations comprise the active compound dissolved or suspended in one or more media such as mineral oil, petroleum, polyhydroxy alcohols or other bases used for topical formulations. The addition of other accessory ingredients as noted above may be desirable.

Further, the present invention provides liposomal formulations of the progestin metabolite and salts thereof. The technology for forming liposomal suspensions is well known in the art. When the progestin metabolite or salt thereof is an aqueous-soluble salt, using conventional liposome technology, the same may be incorporated into lipid vesicles. In such an instance, due to the water solubility of the compound or salt, the compound or salt will be substantially entrained within the hydrophilic center or core of the liposomes. The lipid layer employed may be of any conventional composition and may either contain cholesterol or may be cholesterol-free. When the compound or salt of interest is water-insoluble, again employing conventional liposome formation technology, the salt may be substantially entrained within the hydrophobic lipid bilayer that forms the structure of the liposome. In either instance, the liposomes that are produced may be reduced in size, as through the use of standard sonication and homogenization techniques. The liposomal formulations containing the progesterone metabolite or salts thereof, may be lyophilized to produce a lyophilizate which may be reconstituted with a pharmaceutically acceptable carrier, such as water, to regenerate a liposomal suspension.

Pharmaceutical formulations are also provided which are suitable for administration as an aerosol, by inhalation. These formulations comprise a solution or suspension of the desired progestin metabolite or a salt thereof or a plurality of solid particles of the compound or salt. The desired formulation may be placed in a small chamber and nebulized. Nebulization may be accomplished by compressed air or by ultrasonic energy to form a plurality of liquid droplets or solid particles comprising the compounds or salts.

In addition to the aforementioned ingredients, the compositions of the invention may further include one or more accessory ingredient(s) selected from the group consisting of diluents, buffers, flavoring agents, binders, disintegrants, surface active agents, thickeners, lubricants, preservatives (including antioxidants) and the like.

Having now generally described this invention, the same will be better understood by reference to certain specific examples which are included herein for purposes of illustration only, and are not intended to be limiting of the invention, unless specified.

### EXPERIMENTAL

### Example 1

### Effectiveness of Progesterone Metabolites in Reducing Post-Injury Edema

Surgery

Contusions to the medical frontal cortex (MFC) using a pneumatic impactor device were generated. Animals were anesthetized by injection of Nembutal (50 mg/kg, i.p) and placed in a stereotaxic apparatus, with body core temperature being maintained with a homeothermic heating blanket system. Using aseptic techniques, a midline incision was made in the scalp, and the fascia retracted to expose the cranium. A centered, bilateral craniotomy was made 3 mm anterior to bregma using a 6 mm diameter trephan. After the removal of the bone, the tip of the impactor was moved to AP:3.0; ML:0.0, checked for adequate clearance, retracted to its elevated position and lowered 3.5 mm DV, so it penetrated the cortex 2 mm. The contusion was made at a velocity of 2.25 m/s with a brain contact time of 0.5 seconds. Following this procedure, the wound cavity was thoroughly cleaned and all bleeding stopped before the fascia and scalp, were sutured closed. In all experiments, the rats' group identity was coded with regard to surgery and treatment to prevent experimenter bias during behavioral testing and later histological examination.

All experimental treatments given by injection (progesterone, allopregnanolone, and epipregnanolone) were made in stock solutions using 2-Hydroxypropyl-b-cyclodextrin (HBC; 45% w/v solution in H₂O) as the solvent. These experimental solutions were then diluted 1:1 with sterile water for a final concentration of HBC of 22.5%.

Histopathology Following Cortical Impact

Cortical impact injury to the MFC produces a range of histopathology. For example, at the site of the impact, a large necrotic cavity forms by the seventh day post-injury. Astrogliosis and microgliosis start about 72 hours post-injury and peak about 7 days post injury (Fulop et al. (1992) 22^{nd} Annual Meeting of the Society for Neuroscience 18:178, herein incorporated by reference). By 18 days post-injury there are significant losses of cells in the thalamus (mediodorsal, ventromedial, and ventrolateral thalamic nuclei) accompanied by heavy gliosis (Hoffman et al. (1994) J. Neurotrauma 11:417-31, herein incorporated by reference). The cholinergic magnocellular nucleus in the basal forebrain (nucleus basalis magnocellularis; NBM; the sole source of cholinergic input to the cerebral cortex) at the same time also show significant loss of both choline acetyltransferase-positive cells and Niss1 stained cells (Hoffman et al. (1997) Restorative Neurology and Neuroscience, 11:1-12, herein incorporated by reference). Results indicate that delayed cellular death is occurring several days after injury as revealed by TUNEL labeling in both CA1 and CA3 layers of hippocampus. The data appears to show that the morphology of these cells resembles that of granule neurons.

Group Assignment and Drug Treatment

Sprague-Dawley male rats, approximately 90 days of age at the time of surgery, were used. Rats were housed in individual cages, with a 12:12 light:dark cycle. Food and water were provided ad libitum throughout the experiment. Control rats received sham surgeries and the rest received medial frontal cortical contusions as described in the general methods. The sham-operated controls were given vehicle (cyclodextrin). Contused rats were randomly assigned to control (vehicle), progesterone (4 mg/kg; Sigma), allopregnanolone (4 mg/kg Sigma), or epipregnanolone (4 mg/kg; Sigma). Treatment began one hour after the contusion was produced. Progesterone, allopregnanolone, and epipregnanolone, were given initially intraperitoneally to ensure rapid absorption. Subsequently, a second subcutaneous injection 6 hours post-injury for absorption that is more gradual. Control rats will receive injections of the vehicle by the same route and at the same times. The rats were killed at 24 hours post-injury. This time point was chosen based on previous research indicating that peak edema occurs between 6 and 72 hours post-injury.

Edema Measurements

At 2, 24, and 72 hours the rats were anesthetized, decapitated, and their brains removed quickly from the cranial cavity. The olfactory bulbs, brainstem, and cerebellum were removed and discarded. The brain was placed on a pre-weighed dish and the total weight of the sample was measured. The brains were then placed in a plastic brain mold (Zivic-Miller Labs) and the frontal pole was dissected into two, equal, 4 mm thick sections through the impact area and then separated from the remaining brain tissue. The two sections were placed onto a dry rubber surface and a 3 mm biopsy tissue punch was used to take tissue samples of cortex immediately adjacent to the injured cortex. In addition, two samples from the occipital cortex were also collected and assayed. The tissue samples from each area were pooled and assayed for water content as follows: samples were placed into pre-weighed containers, capped, then immediately weighed to the nearest 0.0001 g. The containers were then uncapped and placed into a vacuum oven and dried at 60° C., 0.3 atm for 24 hours. The containers were then recapped and reweighed to obtain the dry- and wet-weight percentages.

Results

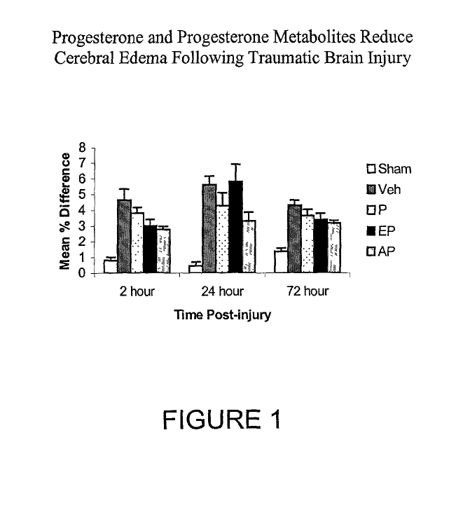

Twenty four hours after injury, all injured rats had significantly (p<0.05) more edema than sham-operates. Allopregnanolone (3×THP) significantly reduced cerebral edema when examined at 2, 24, and 72 hours after injection as compared to rats that were administered only vehicle (FIG. 1). Edema levels in the brain-injured rats given progesterone or epipregnanolone were intermediate between injured controls and the allopregnanolone-treated injured rats. See FIG. 1. None of the experimental substances employed in these experiments proved to be disruptive of recovery; i.e., there were no detectable, negative side-effects that were observed with the current dose/regime of treatment. These data can be taken to indicate that the progesterone metabolites have a moderate effect on reducing cerebral edema at this relatively low dose.

### Example 2

### Effects of Allopregnanolone on Behavioral Recovery Following Traumatic Brain Injury

Surgery

Contusions to the MFC were carried out as described in Example 1.

Group Assignment an Drug Treatment

Adult male rats were given 5 injections of either vehicle (cyclodextrin), progesterone, allopregnanolone, or epipregnanolone (all at 4 mg/kg). These injections started at one hour post-injury with an intraperitoneal injection, and then were repeated at 6, 24, 48, 72, 96, and 120 hours post-injury with subcutaneous injections.

MWM Testing Procedure

The Morris Water Maize (MWM) consisted of a circular tank with a diameter of 133 cm filled with opaque water (20±1° C.; non-toxic Artista™ nontoxic white tempura paint) to a depth of 64 cm (23 cm from top of tank). A platform (11 cm×11 cm) was submerged to a depth of 2 cm and placed approximately 28 cm from the wall of the pool in the center of the northeast quadrant. The position of the platform will remain constant throughout the experiment. MWM testing began seven days after surgery. Each animal was tested for a total of 10 days (2 trials per day) in two 5-day blocks. At the start of each trial, the experimenter placed a rat into the pool at one of four starting positions, and at the same time, activate the computer-interfaced camera tracking system. Each rat was allowed to swim in the pool until it reached the platform or until 90 seconds has passed. If the rats did not find the tile platform in 90 seconds, they were physically guided to the platform. Once a rat found the platform, it was left there for 20 seconds and then removed from the pool for an intertrial interval of 20 seconds. Each rat was then placed in the pool at another start position and the procedure described earlier was repeated. The performance of each rat was measured in terms of latency to platform, length of path to platform, and swim strategy, i.e., percent of total time spent in the outer versus inner annuli.

Results

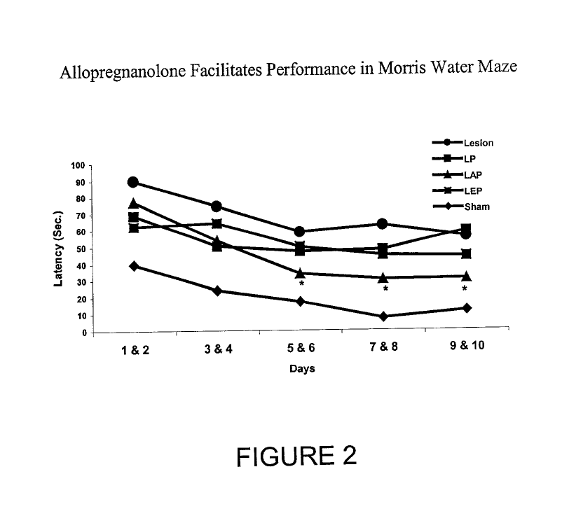

The learning curve for the water maze is measured by comparing the slope of each learning curve to determine if administration of allopregnanolone changed the rate of learning. Rats given allopregnanolone significantly outperformed the injured rats given vehicle on the last three blocks (each block equals 2 days of trials) of testing. See FIG. 2. On all days of testing, the group given allopregnanolone had better performance scores on the spatial learning task than the other groups we examined. Specifically, logarithmic regression demonstrated that the slope of the allopregnanolone treated group was almost twice that of the injured rats that received only vehicle. This indicates that injured rats treated with allopregnanolone learned at a rate nearly twice that of injured rats that received vehicle only. These results can be taken to indicate that the 5 days of treatment with 4 mg/kg injections of allopregnanolone, enhanced behavioral recovery after severe, bilateral contusions of the frontal cortex in rats. Therefore, rats with bilateral frontal cortical contusions given allopregnanolone learned a spatial navigation task more rapidly than untreated controls.

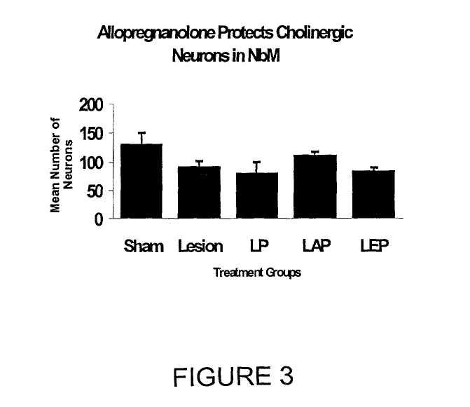

To determine the relationship between neural cell numbers and behavior, the number of neurons and glia were counted to determine the correlative relationship between the survival of these cells and behavioral performance (FIG. 3). Specifically, histological analysis of the number of CHAT positive cells in the nucleus basalis magnocellularis revealed that the allopregnanolone-treated rats had more remaining viable neurons in this structure than untreated controls. See FIG. 3. There were no differences in spontaneous motor behavior or in necrotic cavity size.

### Example 3

### Ability of Allopregnanolone to Decrease Inflammatory Immune Reactions

By reducing the inflammatory response to injury, a substantially reduction in brain swelling and intracranial pressure can follow. Another consequence of reducing the inflammatory immune response is that less neurotoxic substances (e.g., free radicals and excitotoxins) will be released. Reducing the release of neurotoxins from immune cells will result in greater neuronal survival and behavioral recovery after traumatic brain injury by reducing oxidative stress.

I. Increase in mRNA Inflammatory Cytokines After TBI

We have shown that progesterone and its metabolite allopregnanolone reduce cerebral edema and improve spatial performance in a rodent model of traumatic brain injury, but the specific mechanisms leading to recovery are not known. We do know however, that in addition to edema and cell loss, TBI leads to significant increases in inflammatory cytokines (e.g., IL-1β and TNFα), which in turn contribute to cerebral edema and neural cell death. Recently, progesterone and allopregnanolone have been shown to prevent cell death in vitro by authenticating the release of the inflammatory cytokines. This experiment was designed to determine whether the administration of progesterone or allopregnanolone could affect the expression of IL-1β and TNFα after TBI.

Procedure

Adult male SD rates received either bilateral prefrontal cortical contusions or sham surgeries. The neurosteroid injections were given at 1 hr and 6 hr after the contusions and continued once a day for up to 5 consecutive days post-injury. At 3 hr, 8 hr, 12 hr and 6 days post-injury, the rats were killed and their brains were processed for mRNA extraction. Expression of mRNA for IL-1β and TNFα were assessed using real-time quantitative PCR.

Results

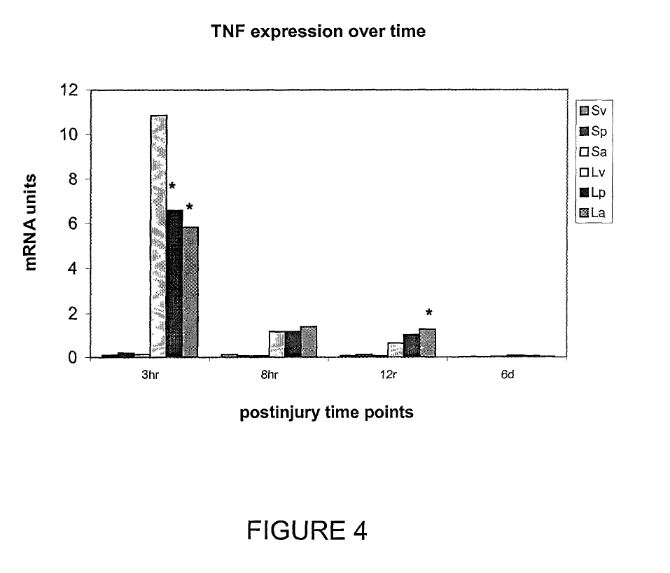

The results from this study indicate that at 3 hours and 6 days post-injury, progesterone and allopregnanolone reduced the expression of both IL-1β and TNFα. See FIGS. 3 and 4. Allopregnanolone, but not progesterone, enhanced IL-1β expression at 8 hrs (FIG. 4), and TNFα expression at 12 hours (FIG. 3) post-injury. Our findings can be taken to suggest that while progesterone and allopregnanolone do not prevent the expression of IL-1β and TNFα, the treatments do delay the synthesis and level of activity for these inflammatory cytokines. Such action could significantly reduce the pathology and behavioral symptomology that often accompanies moderate to severe traumatic brain injuries.

II. Additional technical objectives to be achieved in this experiment, with the proposed treatments in injured rats, would be to: 1) reduce the numbers of inflammatory immune cells (OX42-positive cells) and astrocytes (GFAP-positive cells); 2) reduce the loss in ChAT-positive and COX-positive neurons; 3) reduce the number of TUNEL-positive and MnSOD-positive neurons; and 4) increase the intensity of succinate dehydrogenase and cytochrome oxidase activity.

A. Group Assignment and Drug Treatment

Male Sprague-Dawley rats approximately 90 days of age at the start of the study will be used. The rats will be housed individually in hanging rack-mounted cages on 12:12 light:dark schedule, with food and water available ad libitum throughout the experiment. Prior to surgery, the rats will be assigned to either the sham or the contusion groups. Both groups will then be randomly assigned to either the control (vehicle) or the progesterone (4 mg/kg) condition the allopregnanolone (4 mg/μg) or epipregnanolone (4 mg/kg) and to a survival time (6 hours, 24 hours, 72 hours, 7 days, 14 days, and 28 days). Surgical and drug protocols will follow the procedures described in Example 1.

B. Histology

At given survival times animals will be killed and their brains processed for histology as described herein. Both experiments will use adjacent sections from the same rats; with nine series of sections being collected from each rat brain. Five different series will be stained with antibodies for MnSOD, cytochrome oxidase subunit IV, ChAT, OX42, and GFAP. Two series will undergo histochemical reaction for either succinate dehydrogenase or cytochrome oxidase. One series will undergo in situ hybridization following the TUNEL method. The final series will be reserved for general cell counts with thionin.

C. Immunocytochemistry

For the labeling of viable magnocellular neurons, antibodies to ChAT (monoclonal; Boehringer-Mannheim), MnSOD (polyclonal; Calbiochem), and Cytochrome oxidase subunit IV (monoclonal; Molecular Probes) will be used. For labeling of reactive astrocytes, microglia/macrophages, antibodies to glial fibrillary acidic protein (GFAP) (monoclonal; Boehringer-Mannheim) and to OX42 (monoclonal; Seratec) will be used, respectively. The tissue will then be processed as described in Example 4.

D. Histochemistry

To visualize cytochrome oxidase activity, the following histochemical procedures will be used. The tissue sections will be washed three times in 0.1 M Hepes buffer, pH 7.4. The sections will be then incubated in the dark at 37° C. for 50 minutes with a solution containing Hepes buffer at pH 7.4, cytochrome c, DAB, sucrose, and nickel ammonium sulfate. The reaction will be stopped with three washes in 0.1 M Hepes buffer. The sections will then be dehydrated and cover-slipped.

To visualize succinate dehydrogenase activity, tissue sections will be washed three times in 0.06 M phosphate buffer, pH 7.0. The sections will then be incubated in phosphate buffer containing nitro blue tetrazolium and disodium succinate at 37° C. for 60 minutes. The reaction is stopped by washing the sections in neutral, buffered 4% formaldehyde for 10 minutes. The tissue sections will then be dehydrated and cover-slipped.

E. Detection of TUNEL-Positive Cells

Histological localization and presence of apoptotic cells in the NBM, hippocampus, LMDN, and the cortical areas proximal to the impact site will be examined using an in situ Cell Death Detection Kit (Boehringer Mannheim). The following is the method for frozen tissue. After three washes in PBS the formalin-fixed tissue sections, the slides will be placed in a plastic jar containing 200 ml 0.1 M citrate buffer, pH 6.0, and 750 W (high) microwave irradiation will be applied for 1 min. Following a cooling period in 80 ml distilled water (20-25° C.), the slides will be transferred into PBS (20-25° C.), then immersed for 30 min at room temperature (RT) in 0.1 M Tris-HCl, containing 3% BSA and 20% normal bovine serum, pH 7.5. The slides will then be washed twice in PBS and 50 μl of TUNEL reaction mixture will be placed on each section and incubated for 60 min at 37° C. in a humidified atmosphere. Following three additional washes, endogenous POD-activity will be blocked with 0.3% H₂O₂ in methanol for 10 min at room temperature. After rewashing the tissue in the Tris-BSA-bovine serum mixture, 50 μl Converter-POD, pre-diluted 1:1 in blocking solution will be added, and then the tissue will be incubated for 30 min at 37° C. in a humidified atmosphere. Following three additional washes, the apoptotic cells will be visualized by adding 50 μl of 0.05% DAB substrate solution. After three additional washes in PBS and three in Tris, the tissue will be counter-stained and cover-slipped.

F. Histological Analyses

Quantification of Neurons

Using quantitative stereology counts of ChAT- and MnSOD-positive neurons will be made in the NBM (Michel et al. (1988) J. Microsc. 150:117-36; Gundersen et al. (1986) J. Microsc. 143:3-45; West et al. (1991) Anat. Rec. 231:482-97; all of which are herein incorporated by reference). The NBM measure will be performed on the basis that it has numerous efferents and afferents to the medial frontal cortex. The NBM is demarcated dorsally and medially by the internal capsule (IC), laterally by the caudate-putamen (CPu), and ventrally by the central nucleus of the amygdala (CNA). Counts of thionin stained neurons in the LMDN will be performed as described in Example 4. TUNEL-positive cells will be counted under 40× light microscopy and determined to be apoptotic when the cells are TUNEL positive and meet the anatomical characteristics of apoptotic cells. Cells will be assessed for morphology characteristic of apoptosis and staining according to Gold et al. (1994) Lab. Invest. 71:219-25, herein incorporated by reference. The following changes will be considered to represent programmed cell death: 1) condensation of chromatin and cytoplasm (apoptotic cells); 2) cytoplasmic fragments with or without condensed chromatin (apoptotic bodies); and, 3) chromatin fragments (micronuclei). Cell counts will be performed in the hippocampus, NBM, LMDN, and cortical areas proximal to the site of injury, using the following stereological procedure exemplified for the LMDN. Data will be evaluated for both total number of TUNEL-positive cells and for those meeting the characteristics of an apoptotic cell.

G. Quantification of Reactive Glia and Immune Responsive Cells

Using quantitative stereology counts of GFAP- and OX42-positive cells will be made in the NBM and LMDN. These are areas known to have both significant neuronal degeneration and gliosis after MFC contusions. Quantification will follow the same stereological procedures that are described in Example 4.

H. Cellular Microdensitometry for Mitochondrial Enzymes

The NBM, LMDN, hippocampus, and cortical areas proximal to the site of injury will be analyzed by cellular microdensitometry at the brain levels described above. Images of the nuclei will be digitized for offline analysis by computer aided densitometry, using Image-Pro® Plus by Media Cybernetics running on a Microsoft Windows 98™/Pentium II computer. On each captured image, 3 density measurements of the internal capsule will be taken, averaged, and used for background subtraction. Then individual neurons with a completely visible cell body will be selected and individual densitometric readings will be taken. Each cellular reading will then be corrected for background and averaged for each level.

### Example 4

### The Effectiveness of Allopregnanolone in Promoting Neuroprotection and Behavioral Recovery Following a Traumatic Brain Injury

To determine if the progestin metabolites, such as, allopregnanolone and epipregnanolone are effective at reducing secondary injury caused by traumatic brain injury, the cognitive and sensorimotor deficits after bilateral impact of the frontal cortex were investigated. It will further be determined whether the two progestin metabolites can increase neuronal survival and reduce the inflammatory immune reaction caused by traumatic brain injury by assaying for the neuroprotection, gliosis, and behavioral recovery following a traumatic brain injury using the various assays described below.

Group Assignment and Drug Treatment

Sprague-Dawley male rats, approximately 90 days of age at the time of surgery, will be used. Rats will be housed in individual cages, with a 12:12 light:dark cycle. Food and water will be provided ad libitum throughout the experiment. Sixteen rats will receive sham surgeries and the rest will receive medial frontal cortical contusions as described in the general methods. The sham-operated controls will be given vehicle (HBC; Sigma). Contused rats will be randomly assigned to control (vehicle), progesterone (4 mg/kg), epipregnanolone (1, 4, or 16 mg/kg) and allopregnanolone (1, 4, or 16 mg/kg). Treatment will begin one hour after the contusion is produced. Progesterone, allopregnanolone, and epipregnanolone, will be initially given intraperitoneally to ensure rapid absorption. This will be followed by subcutaneous injections 6 hours post-injury for absorption that is more gradual, and then additional injections will be given once a day for the next five days. Control rats will receive injections of the vehicle by the same route and at the same times. While beneficial effects can be observed within two hours after just one injection of progesterone, additional doses are being used to protect the animals from the secondary wave of edema that may occur over the first several days post-injury. Surgery and testing will be conducted by forming squads of 12 rats, with one rat from each of the experimental groups (see below) selected for the squad.

“Tactile Adhesive Removal”

On the 7th and 27th days post-injury, the rats will be assessed on their responsivity to focal somatosensory stimuli by requiring them to remove sticky paper from their forelimbs. Pairs of circular adhesive papers will be attached to the distal-radial areas of each forelimb and the animal will be returned to its home cage while the investigator holds the forepaws apart and keeps them away from the rat's mouth. The rats' latencies to remove the stimuli with their mouth will be recorded. The maximum length of a “test” trial will be 2 minutes, with each rat receiving four trials with 2 minute intertrial intervals. If the rats do not remove the adhesive disks after 2 minutes, they will be removed by the experimenter.

Histology

At 28 days post-injury, animals will be given an i.p. overdose of pentobarbital (75 mg/kg) then transcardially perfused with phosphate buffered saline (PBS), followed by 4% paraformaldehyde in phosphate buffer. Brains will be removed and post fixed in 4% paraformaldehyde for 1 hr, soaked in 20% sucrose in 0.1 M phosphate buffer at 4° C. for 3 days, frozen on dry ice, and coronally sectioned at 20 μm on a cryostat. Every eighth section will be taken for Niss1 staining with thionin. These sections will be used for lesion reconstruction and general neuronal counts. Three additional series will be labeled with antibodies to ChAT, OX42, and GFAP.

Immunocytochemistry

For the labeling of viable magnocellular cholinergic neurons in the nucleus basalis magnocellularis (NBM), antibodies to ChAT (monoclonal; Boehringer-Mannheim) will be used. For labeling of reactive astrocytes and microglia/macrophages in the NBM, hippocampus, lateral mediodorsal thalamic nuclei and areas around the impact site, antibodies to glial fibrillary acidic protein (GFAP) (monoclonal; Boehringer-Mannheim) and to OX42 (monoclonal; Seratec) will be used, respectively. Tissue sections for immunocytochemistry will be washed in TBS 4×15 min and incubated in endogenous peroxidase inhibitor for 10 min (3% H₂O₂ in TBS). Following a 3×10 min wash in TBS, tissue will be incubated in 10% NGS-TBS 0.1% Triton X-100 (TBS/TX) blocker for 1 h. Primary antibodies will be diluted in 10% NGS-TBS/TX, applied to the tissue, and incubated on a shaker at 4° C. for 48 h. Tissue will be washed 3×10 min in TBS, and incubated for 1 h in the appropriate biotinylated secondary antibody directed against the host animal for the primary antibody (Jackson ImmunoResearch).

Following a 3×10-min wash in TBS, the antibody signal will be associated with a chromogen by incubating with HRP conjugated avidin (A-HRP) which binds the biotinylated secondary antibody. After washing, tissue will be incubated in A-HRP that in turn binds the biotinylated secondary antibody in multiples. The bound HRP will then be visualized by 3,3′ diaminobenzidine tetrachloride (DAB) incubation in the presence of H₂O₂. The reaction will be halted by washing in TBS. Tissue will be mounted on gel coated glass slides, dried at room temperature overnight, dehydrated in alcohol, cleared in xylene, and coverslipped with Shandon-Mount.

Detection of TUNEL-Positive Cells

Histological localization and presence of apoptotic cells will be examined using an in situ Cell Death Detection Kit, POD and the method for frozen tissue (Boehringer Mannheim). After three washes in PBS the formalin-fixed tissue sections, the slides will be placed in a plastic jar containing 200 ml 0.1 M citrate buffer, pH 6.0, and 750 W (high) microwave irradiation will be applied for 1 min. Following a cooling period in 80 ml distilled water (20-25° C.), the slides will be transferred into PBS (20-25° C.), then immersed for 30 min at room temperature (RT) in 0.1 M Tris-HCl, containing 3% BSA and 20% normal bovine serum, pH 7.5. The slides will then be washed twice in PBS and 50 μl of TUNEL reaction mixture will be placed on each section and incubated for 60 min at 37° C. in a humidified atmosphere. Following three additional washes, endogenous POD-activity will be blocked with 0.3% H₂O₂ in methanol for 10 min at room-temperature. After rewashing the tissue in the Tris-BSA-bovine serum mixture, 50 μl Converter-POD, pre-diluted 1.1 in blocking solution will be added, and then the tissue will be incubated for 30 min at 37° C. in a humidified atmosphere. Following three additional washes, the apoptotic cells will be visualized by adding 50 μl of 0.05% DAB substrate solution. After three additional washes in PBS and three in Tris, the tissue will bc counter-stained and cover-slipped.

Histological Analyses

Quantification of Neurons

Using quantitative stereology counts of ChAT-positive neurons will be made in the NBM (Pover et al. (1993) J. Neurosci. Methods 49:123-31; Michel et al. (1988) J. Microsc. 150:117-36; Sterio, D. C. (1984) J. Microsc. 134:127-36; Gundersen et al. (1986) J. Microse. 143:3-45; all of which are herein incorporated by reference). The NBM measure will be performed because the structure has numerous efferents and afferents to the medial frontal cortex. The NBM is demarcated dorsally and medially by the internal capsule (1C), laterally by the caudate-putamen (CPu), and ventrally by the central nucleus of the amygdala (CNA). Counts of thionin stained neurons will be made in the lateral part of the mediodorsal nucleus of the thalamus (LMDN). The LMDN measures will be taken because this Structure has reciprocal connections with the medial frontal cortex. In addition, previous studies with this injury model demonstrate that there is significant loss of these neurons and that progesterone can significantly reduce this neuronal loss (Roof et al. (1999) Exp. Neurol. 129:64-9). The LMDN is demarcated dorsally by lateral habenula (LHb), laterally by the central lateral nucleus (CL), ventrally by the central medial nucleus, and medially by the mediodorsal nucleus (MDN). In addition, TUNEL-positive cells will be counted in the CA1 and CA3 layers of the hippocampus in addition to NBM and LMDN.

For the estimate of the reference volume (V_{(Ref)}), the Cavalieri method will be used (Michel et al (1988) J. Microsc. 150:117-36. Using a low magnification of 4×, the mean reference volume will be estimated for the LMDN. A scale in an ocular micrometer, calibrated with the aid of a 0.01 mm objective micrometer will be used. For each animal, three equally spaced sections will be selected, with the first section starting at a randomly determined number between 1 and k. Using the calibrated ocular micrometer, the width and length of each designated anatomical area for each section will be measured at 3 separate points to produce a mean surface area for that section. These means will be averaged for all 3 sections to determine the estimated two dimensional mean surface area of each structure. The reference volume will then be calculated with the formula V_{(ref)}−ã×t×s, in which “ã” is the mean surface area, “t” is the section thickness, and “s” is the number of sections.

Due to the thickness of the sections (20 μm), and the brain's dissection into three separated series, the optical dissector method will be used for particle counts. The dissector height will be determined by calibrating the microscope's microscrew empirically by measuring the height of its subdivisions at various magnifications with sections of known thickness. This calibration of focusing depth allows for precise and easy movement between the reference and the look-up sections. A dissector height will then be used for all numerical density counts. The dissector volume will be determined by the formula V_{(Dis)}=ã_{(Dis)}×h, in which “ã_{(Dis)}” is the mean area of the reference sections and “h” is the dissector height. For each animal, using a 5×5 (0.16 mm²) grid in the eyepiece, the number of tops will be counted for each area under 40× magnification in each of the 3 chosen reference sections.

### Example 5

### Effects of Stress-Related Hormones on the Ameliorative Effects of Neurosteroids

The detrimental effects of corticosteroids on the sensitivity to excitotoxicity and on synaptic plasticity after brain injury is well documented. See, for example, Goodman et al. (1996) J. Neurochem. 66:1836-44; Supko et al. (1994) Eur. J. Pharmacol. 270:105-13; Scheffet al. (1986) Exp. Neurol. 93:456-70; DeKosky et al. (1984) Neuroendocrinology 38:33-8; all of which are herein incorporated by reference. It is unknown how effective progestin metabolites are when administered under high levels of circulating stress hormones, e.g., corticosterone. The goal of this experiment is to determine effectiveness of the most effective neurosteroid (i.e., progesterone, allopregnanolone, or epipregnanolone) in the presence of high levels of corticosterone in both males and females. To evaluate this interaction, a restraint stress will be used to mimic the hormonal milieu associated with high stress levels. Male and female rats will be subjected to chronic restraint stress before subjecting them to traumatic brain injury. This method is expected to simulate physiological stress, thus allowing us to investigate the interaction of this variable with progestin treatment of traumatic brain injury. Therefore, the actions that elevated levels of corticosterone have on the ameliorative effects of progestins after traumatic brain injury will be investigated. In order to assess this interaction the same physiological and anatomical variables as described in our earlier projects will be examined. These will include behavioral recovery, neuronal survival, inflammatory immune response, and cerebral edema assays as described in Examples 1, 2, 3, and 4.

### Example 6

### Effects of Progesterone on Necrotic Damage and Behavioral Abnormalities Caused by TBI

Methods

Male Sprague-Dawley rats (300 g) were housed individually in wire cages and kept on a reverse light-dark cycle (0800-2000 h). Animals were assigned to one of four groups: (1) lesion (n=7); (2) lesion+3 days progesterone (LP3; n=7); (3) lesion+5 days progesterone (LP5; n=7); and (4) Sham (n=8). All procedures involving animals conformed to guidelines set forth in the Guide for the Care and Use of Laboratory Animals (U.S. Department of Health and Human Services, Pub no. 85-23, 1985) and were approved by the Emory University Institutional Animal Care and Use Committee.

Bilateral contusions of the medial prefrontal cortex were created by a pneumatic impactor device as previously described [40]. Briefly, rats were given anesthetized with ketamine/xylazine (90 mg/kg;10 mg/kg) and placed in a stereotaxic apparatus. A craniectomy (diameter 6 mm) was made over the midline of the prefrontal cortex with its center 1.5 mm AP to bregma. After removal of the bone, the tip of the impactor (diameter 5 mm) was moved to +3.0 mm AP; 1.0 mm ML (from bregma), and checked for adequate clearance. Trauma was produced by pneumatically activating the piston to impact −2.0 mm DV (from dura) at a velocity of 3 m/s with a brain contact time of 0.5 seconds.

Progesterone was dissolved in peanut oil (Sigma; 4 mg/kg) and injections were given at 1 and 6 hours post-injury and then once per day for either 3 or 5 consecutive days. Control animals received injections of vehicle at similar time-points. Animals were coded with regard to surgery and treatment to prevent experimenter bias during behavioral testing and histological examination.

Twenty-one days after surgery, animals were perfused with 100 ml 0.1 M phosphate-buffered saline (PBS; pH 7.4) followed by 400 ml 4% paraformaldehyde in 0.1 M phosphate buffer (PB; pH 7.4). Following cryoprotection in 30% sucrose, coronal 40-μm-thick sections were cut on a freezing microtome, immediately mounted on gel-coated slides and stained for Niss1 with thionine to determine placement and extent of the injury.

Mean area measurements of lesion size were quantified from sections at 15 rostral-caudal levels spaced 300 μm apart. The perimeter of the necrotic cavity (including injured penumbra) was traced on digitized images using the Jandel Scientific SigmaScan software calibrated to calculate the area in mm² for each level traced. Perimeters of the striatum and the lateral ventricles were also traced and mean areas were quantified from 7 rostral-caudal levels (300 μm apart).

Cell counts were done on an Olympus BH-2 microscope equipped with an eyepiece micrometer grid (sample area=40 μm² at ×400 magnification). Bilateral cell counts of Niss1-stained neurons were made on 3 separate sections in each of the following areas: (1) STR (+1.8 to +1.2 mm AP), (2) GP (−0.3 to −1.2 mm AP), (3) DMN (−2.3 to −2.9 mm AP), and (4) VMN (−2.3 to −2.9 mm AP). Only cells with neuronal nuclei and intact membranes were counted as neurons.

Experienced individuals who were blind to treatment conditions of the study conducted all histological and behavioral analyses. All data were tested for normality and homoschedasticity before being analyzed by parametric analysis of variance (ANOVA). MWM results were analyzed using separate mixed-factorial (4 groups×5 days) analysis of variance (ANOVA) on each of the two 5-day testing periods (acquisition and retention respectively). Results of the BSN task were analyzed using the mixed-factorial ANOVA (4 groups×2 post-injury trials). Histological comparisons on mean densitometry recordings, area measurements, and cell counts were made using a one-way ANOVA. All between-group comparisons were made using multiple Tukey post-hoc tests (p<0.05) when the overall ANOVA was significant (p<05) between groups. Pearson r coefficients were calculated to determine whether significant correlations could be detected between histological (e.g., lesion size and cellular density) and behavioral parameters (e.g., acquisition and retention of the MWM task and measures of sensory neglect).

Beginning one week after surgery, spatial learning ability was assessed in the Morris water maze (MWM) task described previously. Each animal was tested for a total of 10 days in two 5-day trial blocks (acquisition and retention respectively). Animals were placed in the pool (nose facing the pool-wall) at one of four randomly determined starting positions (e.g.: N, S, E, W). Each rat was allowed to swim freely in the pool until it found the hidden platform or until 90 seconds had elapsed. If an animal did not find the platform in 90 seconds, he was manually guided to it. Once on the platform, animals were allowed to rest for 10 seconds and then removed from the pool and placed near a heat lamp for warmth. Each rat was given two trials per day with a 20-second intertrial interval (ITI). The dependent measures for this task were latency to find the hidden platform and swim strategy (e.g., percent of time spent in the inner vs. outer annuli). Swim speed measures were recorded daily in order to delineate motor dysfunction from learning impairment.

Measures of attentional abilities, using a bilateral sensory neglect (BSN) task, were recorded one day prior to surgery (baseline) and on postsurgical days 6 and 20. Pairs of circular adhesive papers (2 cm dia) were attached to the distal-radial areas of each forepaw and the rats' latencies to remove the stimuli were recorded. Each rat was given four trials (2-min ITI) per testing period with a maximum trial length of 2 minutes. If the rats did not remove the adhesive disks within the standard time, a total latency of 2 minutes was recorded for that trial.

Results

Histology. In most animals, necrotic tissue was primarily restricted to the medial prefrontal and cingulate cortex. However, in some cases, more severe tissue damage extended into the corpus callosum and the most dorsal aspects of the medial septum and striatum (Data not shown). A significant main effect on necrotic cavity formation was observed between the three injured groups, (F_{2,19}=3.57, P<0.05). Tukey post hoc analysis revealed a dose-dependent reduction in necrotic cavity formation. Data not shown. Notably, all animals that were given progesterone tended to have smaller lesions compared to injured animals that were given vehicle injections. However, only 5 days of progesterone resulted in significant reductions in overall necrotic cavity formation (P<0.05). We also observed enlargement of the lateral ventricles in all injured groups as compared to control animals (F_{3,25}=5.28, P<0.01) but progesterone did not have any effect on this measure. Data not shown. No between-group differences were shown on measures of mean striatal area.

One-way ANOVA revealed a main effect of mean cellular density between groups on counts taken in the STR (F_{3,25}=15.58, P<0.01), GP (F_{3,25}=4.47, P<0.01), DMN (F_{3,25}=5.37, P<0.01), and VMN (F_{3,25}=8.68, P<0.01). Results of Tukey post hoc tests showed that both LP3 and LP5 treatments resulted in a significant reduction of injury-induced neuronal loss in all brain regions examined. However, 5 days of progesterone was more effective than 3 days at attenuating neuronal loss in the VMN, the area most distal to injured penumbra. Data not shown.

Behavioral Testing. In the MWM task, all of the injured groups displayed deficits in spatial learning performance as compared to control animals during the initial 5-day acquisition phase (F_{3,25}=19.45, P<0.01). However, Tukey post hoc tests detected improved spatial learning performance in LP5, but not LP3, animals during the second 5-day trial block (F_{3,25}=6.76, P<0.01). Data not shown.

ANOVA revealed a significant main effect on swim patterns during acquisition (F_{3,25}=28.23, P<0.01) and retention (F_{3,25}=12.25, P<0.01) of the MWM task. Data not shown. All the injured animals displayed sustained thigmotaxic (wall-hugging) swim patterns during the first 5-day MWM trial block. But a reduction of thigmotaxic behavior was observed in the LP5-treated animals in the last 2 days of the second phase of MWM testing (P>0.05 compared to controls) corresponding with the reduction in latency to find the platform observed in this group. There were no between-group differences on swim speed measurements on any day of testing.

There were no between-group differences on baseline measures of sensory neglect recorded one day prior to surgery. A significant main effect between groups (F_{3,25}=6.17, P<0.01) was observed in results of the BSN task following controlled cortical contusion to the medial prefrontal cortex. Tukey post hoc analysis showed that only the LP3-treated animals were impaired on this task compared to control animals at both 6 and 20 days post injury Data not shown).

We also detected significant correlations between histological measures and performance in the MWM task. Specifically, there was a positive correlation between necrotic cavity formation and improved MWM performance during the second 5-day trial block, suggesting that smaller lesions resulted in improved retention of this task (r₂₁=+0.44, P<0.05). Similarly, we observed a negative correlation between cellular density and spatial learning performance during the second phase of MWM testing (r₂₁=−0.50, P<0.05) which indicates that progesterone-mediated neuronal sparing allowed for greater functional recovery (data not shown). Finally, we did not observe any significant correlations between either lesion size or cellular density and measures of sensory neglect.

Summary

The reduction of the injury-induced necrotic cavity formation provides evidence that a post-injury neurosteriod intervention might reduce lesion volume following TBI in this animal model. In the present study, we observed a dose-dependent reduction in necrotic cavity formation in progesterone treated animals. Specifically, while the necrotic cavities in the brains of animals treated with only 3 days of progesterone (LP3) tended to be smaller than in the brains of injured animals, only the 5-day treatment regimen (LP5) resulted in significantly smaller lesions. Our study now provides the first evidence that progesterone may also attenuate TBI-induced tissue loss.

In our study, progesterone also protected against secondary cell loss in brain regions both proximal (e.g., STR) and distal (e.g., GP, DMN, and VMN) to the zone of injury. Interestingly, in the present study, both 3 and 5 days of progesterone treatment reduced neuronal loss in the STR, GP, and DMN, but only LP5-treatments produced significant reductions in cell loss of the VMN compared to untreated controls.

And finally, in the present study, all injured groups were impaired on the acquisition phase of MWM testing. The LP5 animals showed clear improvement, albeit not to control levels, in spatial performance during the retention phase of this task. Significant correlations were found between neuropathological parameters (e.g., necrotic cavity formation and neuronal sparing) and MWM performance demonstrating that progesterone-mediated reductions in lesion size cell death resulted in concomitant reductions in latency to find the platform.

### Example 7

### Dosage Response Curves for the Behavioral Recover Following TBI Upon Administration of Progesterone in a Cylcodextrin Vehicle

Methods

Surgery to induce a traumatic brain injury was performed as outlined in Example 1. Behavior testing using the Morris Water Maize was performed as outlined in Example 1 and the methods for the tactical adhesive removal were performed as outlined in Example 4.

Results

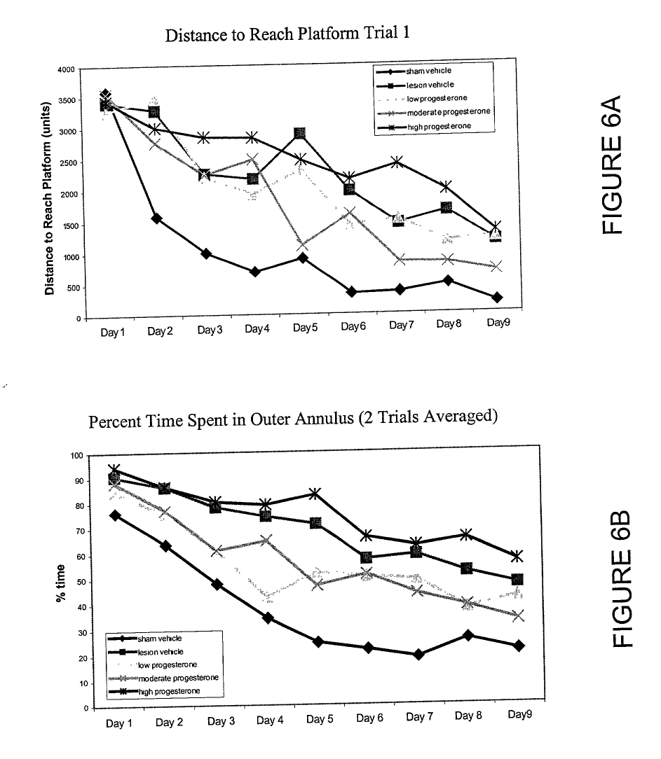

FIGS. 6A and 6B demonstrate that low and moderate doses of progesterone (8 mg/kg & 16 mg/kg in a cyclodextrin-containing vehicle) produced consistent improvement in Morris water maze performance, whereas the high dose of progesterone (32 mg/kg in a cyclodextrin-containing vehicle) did not show any beneficial effect.

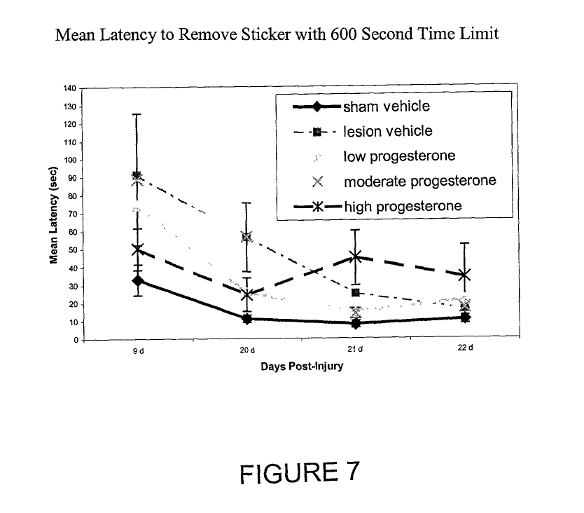

The sticker removal task is a test for sensory neglect which is a primary deficit for frontal injury. In this task all doses initially produce behavioral recovery, however, the group receiving the high dose of progesterone degraded to lesion control levels and the moderate dose, which was initially at lesion control levels improved to sham levels by day 21 post-injury. See FIG. 7.

### Experiment A

### Are Progestins Neuroprotective after Traumatic Brain Injury in Situations of Chronic Stress?

Group Assignment and Drug Treatment

Sprague-Dawley male and female rats approximately 90 days of age at the start the study will be used. The rats will be housed individually in hanging rack-mounted cages on 12:12 light:dark schedule, with food and water available ad libitum throughout the experiment. Prior to surgery, the rats will be assigned to either the sham or the contusion groups and to either chronic restraint stress or to no stress groups. Both groups will then be randomly assigned to either the control (vehicle) or the neurosteroid (most effective neurosteroid at most effective dosage as determined from the examples described above. Surgical and drug protocols will follow the procedures described in Example 1.

Chronic Restraint Stress

Rats that will receive chronic stress will be subjected to 6 hours of forced restraint at the same time each day (10:00 h to 16:00 h) in their home cages for 21 days prior to injury. The rats will be restrained in plastic animal injection holders. Blood samples for corticosterone serum assays will be from the tail vein twice per day at 9:00 h and 19:00 h on days 1, 5, 14, and 21 during the pre-injury stress period. The samples will be centrifuged and the serum will be stored at −80° C. until processing for radioimmunoassay (RIA). This assay will enable a correlation between the physiological ‘levels’ of stress with the subsequent rate and extent of morphological and behavioral recovery.

Blood Assay for Corticosterone

Plasma corticosterone (5 μl) will be measured using the RIA kit of ICN Biomedicals with [¹²⁵I] corticosterone as a tracer. The corticosterone antibody cross-reacts 100% with corticosterone, slightly with desoxycorticosterone (0.34%), testosterone, and cortisol (0.10%), but does not cross-react with the progestins or estrogens (<0.01%). The detection limit of the assay is 0.2 μg/dl.

The MWM testing procedure, histology, immunocytochemistry, and the quantification of neurons and glia will be performed as described in Examples 1, 2, 3, and 4.

### Experiment B

### Effects of Stress on the Progestin-Related Reduction of Cerebral Edema

Group Assignment and Drug Treatment

Sprague-Dawley male and female rats approximately 90 days of age at the start the study will be used. The rats will be housed individually in hanging rack-mounted cages on 12:12 light:dark schedule, with food and water available ad libitum throughout the experiment. Prior to surgery, the rats hill be assigned to either the sham or the contusion groups and to either chronic restraint stress or no stress. Both contusion groups will then be randomly assigned to either the control (vehicle) or the neurosteroid (most effective neurosteroid at most effect dosage as determined in the examples described above). Surgical and drug protocols will follow the procedures described in Example 1. The time points for this experiment were chosen based on previous research indicating that peak edema occurs between 6 and 72 hours post-injury. In order to concentrate on the most critical time points we will initially look at 24 and 48 hours, and if there are differences in the rats exposed to stress, we will then include 6 and 72 hour time points. For the purpose of objective analyses, animals from each experimental group will be formed into squads by selecting one from each experimental condition to form groups of 12 each.

Chronic Restraint Stress

Those rats that will receive chronic stress will be subjected to 6 hours of restraint stress at the same time each day (10:00 h to 16:00 h) in their home cages. The rats will be restrained in plastic animal injection holders. Blood samples for corticosterone serum assays will be from the tail vein twice per day at 9:00 h and 19:00 h on days 1, 7, 14, and 21 during the pre-injury stress period.

Blood Assay for Corticosterone

Will follow same protocol as described in Experiment A.

Edema Measurements

Will follow the same protocol as described in Example 1.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

That which is claimed:

1. A method of treating a traumatic central nervous system injury, said method comprising administering to a patient in need thereof a therapeutically effective amount of a composition comprising allopregnanolone.

2. The method of claim 1, wherein said injury is a traumatic brain injury.

3. The method of claim 2, wherein said traumatic brain injury results from a blunt force contusion.

4. The method of claim 1, wherein said method reduces edema in the patient following said traumatic CNS injury.

5. The method of claim 1, wherein said method reduces the inflammatory response in the patient following said traumatic CNS injury.

6. The method of claim 1, wherein said method reduces neuronal cell death in the patient following said traumatic CNS injury.

7. The method of claim 1, wherein said allopregnanolone is administered in at least one dosage of about 1 μg/kg to about 50 mg/kg of body weight.

8. The method of claim 7, wherein said allopregnanolone is administered in at least one dosage of about 4 mg/kg of body weight.

9. The method of claim 7, wherein at least one dosage of said allopregnanolone is administered about 0.5 to about 100 hours following the traumatic CNS injury.

10. The method of claim 7, wherein the first dose of the allopregnanolone is administered about 1 hour following the traumatic CNS injury, and a subsequent allopregnanolone dose is administered about 6 hours following the injury.

11. The method of claim 7, wherein the first dose of the allopregnanolone is administered about 1 hour following the traumatic brain injury, a second allopregnanolone dosage is administered about 6 hours following the injury, and subsequent allopregnanolone dosages are administered in 24 hour intervals.

12. The method of claim 1, wherein said allopregnanolone is administered by intraperitoneal, subcutaneous, intravenous or intracerebroventricular administration or any combination thereof.

13. The method of claim 1, wherein said allopregnanolone is administered in a pharmaceutically acceptable carrier.

14. The method of claim 13, wherein said carrier is cyclodextrin.

15. The method of claim 1, wherein said composition further comprises at least one other neurotrophic agent.

16. A method of decreasing neurodegeneration on a population of cells in a subject following a traumatic injury to the central nervous system, said method comprising administering to a patient in need thereof a therapeutically effective dose of allopregnanolone, wherein said dose produces a neuroprotective effect in the patient.

17. The method of claim 16, wherein said traumatic CNS injury is a traumatic brain injury.

18. The method of claim 17, wherein the neurodegeneration is associated with cerebral edema.

19. The method of claim 17, wherein the neurodegeneration is associated with a blunt force contusion.

20. The method of claim 17, wherein the neurodegeneration is associated with an inflammatory response.
